(19) United States
(12) Patent Application Publication
(10) Pub. No.: US 2024/0317831 A1
(43) Pub. Date: Sep. 26, 2024

(54) NK CELL ENGAGER MOLECULES AND METHODS OF USE

(71) Applicant: REGENTS OF THE UNIVERSITY OF MINNESOTA, Minneapolis, MN (US)

(72) Inventors: Martin Felices, Minneapolis, MN (US); Jeffrey S. Miller, Minneapolis, MN (US); Todd R. Lenvik, Minneapolis, MN (US)

(21) Appl. No.: 18/269,803

(22) PCT Filed: Jan. 5, 2022
(86) PCT No.: PCT/US2022/011301

§ 371 Date: Jun. 27, 2023

Related U.S. Application Data
(60) Provisional application No. 63/135,162, filed on Jan. 8, 2021.

(51) Int. Cl. C07K 14/54; A61K 38/00; A61K 39/00; C07K 16/28

(52) U.S. Cl. CPC C07K 14/5434; C07K 16/2803; C07K 16/2827; C07K 16/283; A61K 38/00; A61K 2039/505; C07K 2319/21; C07K 2319/33

(57) ABSTRACT

A compound generally includes an NK cell engaging domain operably linked to the NK cell engaging domain. The NK activating domain generally includes an IL-12-based polypeptide. The NK engaging domain selectively binds to an NK cell and an NK activating domain. In some embodiments, the IL-12 polypeptide can include an IL-12A polypeptide, an IL-12B polypeptide, or both an IL-12A polypeptide and an IL-12B polypeptide. In some embodiments, the NK activating domain includes an IL-12A polypeptide, an IL-12B polypeptide, and a flanking sequence linking the IL-12A polypeptide and the IL-12B polypeptide. In some embodiments, the compound can include a second NK activating domain. In some embodiments, the compound can include ones or more targeting domains.

### CROSS-REFERENCE TO RELATED APPLICATION

This application claims the benefit of U.S. Provisional Patent Application No. 63/135,162, filed Jan. 8, 2021, which is incorporated herein by reference in its entirety.

### GOVERNMENT FUNDING

This invention was made with government support under CA150085 awarded by the Department of Defense and CA111412 awarded by the National Institutes of Health. The government has certain rights in the invention.

### SEQUENCE LISTING

This application contains a Sequence Listing electronically submitted via EFS-Web to the United States Patent and Trademark Office as an ASCII text file entitled “0110-000670WO01_ST25.txt” having a size of 42 kilobytes and created on Jan. 5, 2022. The information contained in the Sequence Listing is incorporated by reference herein.

### SUMMARY

This disclosure describes, in one aspect, a compound comprising that generally includes an NK cell engaging domain that selectively binds to an NK cell and an NK activating domain operably linked to the NK cell engaging domain. The NK activating domain generally includes an IL-12-based polypeptide.

In some embodiments, the compound can further include a targeting domain that specifically binds to a target antigen.

In some embodiments, the IL-12 polypeptide can include an IL-12A polypeptide, an IL-12B polypeptide, or both an IL-12A polypeptide and an IL-12B polypeptide.

In some embodiments, the IL-12A polypeptide includes the amino acid sequence of SEQ ID NO:5 or a functional variant thereof.

In some embodiments, the IL-12B polypeptide includes the amino acid sequence of SEQ ID NO:4 or a functional variant thereof.

In some embodiments, the NK activating domain includes an IL-12A polypeptide, an IL-12B polypeptide, and a flanking sequence linking the IL-12A polypeptide and the IL-12B polypeptide.

In some embodiments, the compound can include a second NK activating domain.

In some embodiments, the compound can include two or more targeting domains.

In another aspect, this disclosure describes a method that includes administering to a subject any embodiment of the compound summarized above in an amount effective to induce NK-mediated killing of a cancer cell.

In another aspect, this disclosure describes a method for stimulating degranulation of NK cells in vivo. Generally, the method includes administering to a subject any embodiment of the compound summarized above in an amount effective to stimulate degranulation of NK cells in the subject.

In another aspect, this disclosure describes a method for stimulating IFNγ secretion by NK cells in vivo. Generally, the method includes administering to a subject any embodiment of the compound summarized above in an amount effective to stimulate IFNγ secretion by of NK cells in the subject.

In another aspect, this disclosure describes a method of rescuing exhaustion of NK cells in vivo. Generally, the method includes administering to a subject any embodiment of the compound summarized above in an amount effective to rescue exhaustion by of NK cells in the subject.

In another aspect, this disclosure describes a method of treating cancer in a subject. Generally, the method includes administering to a subject any embodiment of the compound summarized above in an amount effective for treating the cancer. In some of these embodiments, the method can include administering the compound prior to, simultaneously with, or following chemotherapy, surgical resection of a tumor, or radiation therapy.

In another aspect, this disclosure describes a composition that includes any embodiment of the compound summarized above and a pharmaceutically acceptable carrier.

In some embodiments, the composition can further include an additional therapeutic agent. In some of these embodiments, the additional therapeutic agent comprises altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, vinorelbine, or a therapeutic agent that targets HER2, HER3, or a HER2/HER3 heterodimer complex.

In another aspect, this disclosure describes a method that includes administering to a subject any embodiment of the composition summarized above in an amount effective to induce NK-mediated killing of a cancer cell.

In another aspect, this disclosure describes a method for stimulating degranulation of NK cells in vivo. Generally, the method includes administering to a subject any embodiment of the composition summarized above in an amount effective to stimulate degranulation of NK cells in the subject.

In another aspect, this disclosure describes a method for stimulating IFNγ secretion by NK cells in vivo. Generally, the method includes administering to a subject any embodiment of the composition summarized above in an amount effective to stimulate IFNγ secretion by of NK cells in the subject.

In another aspect, this disclosure describes a method of rescuing exhaustion of NK cells in vivo. Generally, the method includes administering to a subject any embodiment of the composition summarized above in an amount effective to rescue exhaustion by of NK cells in the subject.

In another aspect, this disclosure describes a method of treating cancer in a subject. Generally, the method includes administering to a subject any embodiment of the composition summarized above in an amount effective for treating the cancer. In some of these embodiments, the method includes administering the composition prior to, simultaneously with, or following chemotherapy, surgical resection of a tumor, or radiation therapy.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. IL-12 Killer Engager compound design and general function. An exemplary IL-12 Killer Engager is shown schematically as having three domains: an NK engaging domain, an IL-12 domain, and a targeting domain.

FIG. 2. Effectors (NK cells) were incubated alone or with THP1 (AML cell line) targets at a 2:1 ratio in the absence (no drug) or presence of 30 nM IL-12 Killer Engager (cam161233; SEQ ID NO:1). After five hours cells were stained for degranulation (CD107a) and inflammatory (IFNγ) markers and run on a flow cytometer. Data was analyzed using FLOWJO software (BD Biosciences, San Jose, CA).

FIG. 3. cam161233 (SEQ ID NO:1) binds selectively to NK cells and remains bound without CD16. (A) Peripheral blood mononuclear cells (PBMCs) were either treated with media alone (Resting, open bars) or PMA/Ionomycin (PMA/I activated, filled bars) to evaluate binding of the IL-12 Killer Engager (cam161233; SEQ ID NO: 1) in the presence of CD16. (B) PBMCs were either treated with media alone (Resting, open bar) or PMA/Ionomycin (PMA/I activated, filled bar) to evaluate binding of the IL-12 Killer Engager (cam161233) in the absence of CD16, which is clipped when NK cells are activated with PMA/I (graph on the right). Results indicate preferential binding of NK cells, particularly when CD16 is present.

FIG. 4. IL-12 domain of cam161233 Killer Engager (SEQ ID NO:1) is functional. To evaluate specificity of the Killer Engager compounds and activity of the IL-12 component of the Killer Engager compound, effectors (NK cells) were incubated alone or with CD33-expressing THP1 (AML cell line) targets or CD33-negative Raji (Burkitt's lymphoma) targets at a 2:1 ratio in the absence (No treatment) or presence of 30 nM IL-12 Killer Engager cam161233 (SEQ ID NO:1) or IL-12 Killer Engager (cam161233)+IL-18. The IL-12 Killer Engager+IL-18 acts as a positive control independent of specificity. (A) After five hours cells were stained for degranulation (CD107a) and subjected to flow cytometry. (B) After five hours cells were stained for inflammatory markers (IFNγ) and subjected to flow cytometry.

FIG. 5. In vitro NK cell exhaustion. (A) Schematic illustrating in vitro NK cell exhaustion assay in which K562 (CML line that potently activates natural cytotoxicity on NK cells) cells are added at multiple times over the course of eight days and then cell function (degranulation and inflammatory cytokine production) is assessed on the eighth day by flow cytometry. Briefly, on the eighth day, Effectors (NK cells) were incubated with K562 targets at at 2:1 ratio. (B) After five hours, cells were stained for degranulation (CD107a) and subjected to flow cytometry. (C) After five hours, cells were stained for inflammatory (IFNγ) markers and subjected to flow cytometry.

FIG. 6. Only IL-12 and cam161233 rescues IFNγ production in exhausted cells. Exhaustion assay was carried out and on the eighth day effectors (NK cells) were incubated with THP1 targets at a 2:1 ratio and either IL-12, IL-15, IL-12 Killer Engager cam161233 (SEQ ID NO:1), or IL-15 Killer Engager (161533; SEQ ID NO:24) (30 nM). After five hours, cells were stained for inflammatory (IFNγ) markers and subjected to flow cytometry. Data were analyzed using FLOWJO software (BD Biosciences, San Jose, CA).

FIG. 7. IL-12 Killer Engager (cam161233; SEQ ID NO:1) induced degranulation similarly to IL-15 Killer Engager (cam161533; SEQ ID NO:XX). Exhaustion assay was carried out and on the eighth day effectors (NK cells) were incubated with THP1 targets at a 2:1 ratio and either IL-12, IL-15, IL-12 Killer Engager cam161233 (SEQ ID NO:1), or IL-15 Killer Engager (161533; SEQ ID NO:24) (30 nM). After five hours, cells were stained for degranulation (CD107a) and subjected to flow cytometry. Data were analyzed using FLOWJO software (BD Biosciences, San Jose, CA).

FIG. 8. NK cells were incubated alone or with THP1 cells ⁺/₋ IL-12 Killer Engager (SEQ ID NO:1) for 24 hours and supernatant was collected. Supernatant was then incubated with monocyte-derived dendritic cells (DCs) for five days. (A) Expression of dendritic cell maturation marker CD86 was evaluated by flow cytometry. (B) Expression of dendritic cell maturation marker CD80 was evaluated on by flow cytometry. Data were analyzed using FLOWJO software (BD Biosciences, San Jose, CA).

FIG. 9. B7H3-targeting IL-12 Killer Engager (B7H3-IL12KE; SEQ ID NO:10) induces NK cell activation against solid tumors. Normal donor peripheral blood mononuclear cells in (PBMCs) were incubated alone (top): with no treatment (left), 30 nM IL-12 (center), or 30 nM B7H3-IL12 Killer Engager (SEQ ID NO:10 (right) for a five-hour period to evaluate background activation in the absence of tumor. PBMCs were incubated with a prostate cancer cell line (PC3; bottom left) or an ovarian cancer cell line (OVCAR; bottom right) and 30 nM B7H3-IL12 Killer Engager (SEQ ID NO:10) to determine the ability of the IL12 Killer Engager to specifically induce NK cell activation against B7H3-expressing solid tumors. To measure activation, surface CD107a (a measure of NK cell degranulation) and intracellular interferon gamma (IFNγ; a measure of NK cell inflammatory cytokine production) were evaluated by flow cytometry on gated CD56⁺CD3⁻ NK cells.

FIG. 10. Pooled data showing B7H3-targeting IL12 Killer Engager (B7H3-IL12KE; SEQ ID NO:10) induces NK cell activation against prostate cancer. Normal donor peripheral blood mononuclear cells (PBMCs) were incubated alone (white bars) or with PC3 prostate cancer targets (black bars): with no treatment, 30 nM IL-12, or 30 nM B7H3-IL12KE (SEQ ID NO:10) for a five-hour period to evaluate activation in the absence or presence of tumor. To measure activation, surface CD107a (a measure of NK cell degranulation; left) and intracellular interferon gamma (IFNγ; a measure of NK cell inflammatory cytokine production; right) was evaluated by flow cytometry on gated CD56⁺CD3⁻ NK cells. N=4.

### DETAILED DESCRIPTION OF ILLUSTRATIVE EMBODIMENTS

This disclosure describes multispecific Killer Engager compounds that include an IL-12-based NK activating domain. The Killer Engager compound can be bi-specific, tri-specific, tetra-specific, or larger. For example, in a tri-specific embodiment, the Killer Engager compound includes the IL-12-based NK activating domain, an NK engaging domain, and a targeting domain. In a bispecific embodiment, the Killer Engager compound includes IL-12-based NK activating domain and an NK engaging domain. In a tetra-specific embodiment, the Killer Engager compound can include the IL-12-based NK activating domain, an NK engaging domain, and two targeting domains. Alternatively, a tetra-specific Killer Engager compound can include an NK engaging domain, a targeting domain, the IL-12-based NK activating domain, and a second NK activating domain. Suitable members of each domain are described in detail below.

Killer Engager compounds have been previously described (U.S. Pat. Nos. 11,098,100; 11,098,101; US Patent Application Publication No. 2020/0087369; and International Patent Publication Nos. WO 2021/055342, WO 2021/076545, and WO 2021/247794).

The IL-12 Killer Engager compounds described herein include a recombinant IL-12-based NK activation domain. The Killer Engager compounds described herein specifically target the recombinant IL-12-based NK activating domain to the NK-target (e.g., tumor cell) synapse that is generated by the IL-12 Killer Engager compound. The IL-12-based NK activating domain induces NK-cell-mediated cytotoxicity and enhance antibody-dependent cellular cytotoxicity (ADCC) of tumor targets. Further, the IL-12 Killer Engager compounds can robustly stimulate IFNγ secretion, likely creating a potent inflammatory response. IL-12 activation also can better equip NK cells to bridge the innate and adaptive immune responses through NK-cell-mediated dendritic cell (DC) maturation. Finally, the IL-12 Killer Engagers can rescue NK cell exhaustion.

This disclosure describes, in one aspect, IL-12-based Killer Engager compounds that generally include an IL-12-based NK activating domain, one or more NK cell engager domains, and one or more targeting domains, with each domain operably linked to the other domains. As used herein, the term “operably linked” refers to direct or indirect covalent linking. Thus, two domains that are operably linked may be directly covalently coupled to one another. Conversely, the two operably linked domains may be connected by mutual covalent linking to an intervening moiety (e.g., a flanking sequence). Two domains may be considered operably linked if, for example, they are separated by the third domain, with or without one or more intervening flanking sequences.

The use of an IL-12 Killer Engager compound selectively delivers IL-12 to NK cells, induces degranulation and IFNγ secretion, and increases IFNγ in exhausted NK cells better than comparable IL-15-based Killer Engager compounds.

While described herein in the context of a tri-specific Killer Engager compound that includes an IL-12-based NK activating domain, an anti-CD16 NK engaging domain, and an anti-CD33 targeting domain, the NK Killer Engager compounds described herein can include additional NK activating domains, one or more of any suitable targeting domains, and/or one or more of any suitable NK engaging domains.

The IL-12-based NK activating domain can include wild-type human IL-12, or any variant thereof that includes an amino acid sequence that activates NK cells, promotes sustaining NK cells, rescues NK cell exhaustion, or otherwise promotes NK cell activity (e.g., degranulation or IFNγ production). IL-12 includes two signaling subunits, IL-12A and IL-12B. The IL-12-based NK activating domain includes, or is derived from, human IL-12A and/or IL-12B. As used herein, the term “derived from” refers to an amino acid fragment of IL-12A and/or IL-12B that is sufficient to provide NK cell activating and/or sustaining activity (e.g., induce degranulation, induce IFNγ secretion, and/or increase IFNγ in exhausted NK cells). Thus, the IL-12-based NK activating domain can include the amino acid sequence of human IL-12A (SEQ ID NO:4), human IL-12B (SEQ ID NO:5), or both. When the NK activating domain includes both IL-12A and IL-12B, the NK activating domain can further include additional amino acids such as, for example, a linker or flanking sequence. Suitable flanking sequences that may be used to link IL-12 subunits are described in detail below.

Further, when the NK activating domain includes both IL-12A and IL-12B, these components can be present in any order. Thus, while the exemplary embodiments shown in SEQ ID NO: 1 and SEQ ID NO:2 present IL-12B as toward the N-terminal of the molecule compared to IL-12A, IL-12A can be present toward the N-terminal of the molecule compared to IL-12B.

In embodiments that include more than one NK activating domain, the additional NK activating domain or domains may be provided in series or in any other combination. Each additional NK activating domain can be selected, independently of every other NK activating domain, to include an NK activating cytokine. Further, each NK activating domain can include either the full amino acid sequence of the cytokine or an NK activating fragment of the cytokine, independent of the nature of other NK activating domains included in the IL-12-based Killer Engager compound. Exemplary cytokines on which an additional NK activating domain may be based include, for example, IL-15, IL-18, and IL-21.

For brevity in this description, reference to an NK activating domain by identifying the cytokine on which it is based includes both the full amino acid sequence of the cytokine (or cytokine subunit such as IL-12A or IL-12B), any suitable NK activating fragment of the cytokine (or cytokine subunit), and or a modified version of the cytokine (or cytokine subunit) that includes one or more amino acid substitutions. Exemplary amino acid substitution can include, but are not limited to, conservative amino acid substitutions. A conservative substitution for an amino acid residue within an amino acid sequence may be selected from other members of the class to which the amino acid residue belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Thus, conservative substitutions include, for example, Lys for Arg and vice versa to maintain a positive charge; Glu for Asp and vice versa to maintain a negative charge; Ser for Thr so that a free-OH is maintained; and Gln for Asn to maintain a free-NH₂.

Thus, reference to an IL-12 NK activating domain includes an NK activating domain that includes the full amino acid sequence of human IL-12A, the full amino acid sequence of human IL-12B, a fragment of IL-12A, a fragment of IL-12B, a variant of IL-12A, a variant of IL-12B, or a combination of any two or more of the foregoing. Suitable IL-12A variants and/or IL-12B variants can include one or more amino acid additions, amino acid deletions, or amino acid substitutions that retain IL-12 NK activating activity (e.g., NK cell activation, promotes sustaining NK cells, rescues NK cell exhaustion, or otherwise promotes NK cell activity such as degranulation or IFNγ production). As another example, reference to an “IL-15” NK activating domain (e.g., in the context of an additional NK activating domain) includes an NK activating domain that includes the full amino acid sequence of IL-15, IL-15 linked to IL-15 receptor alpha (in whole or as a sushi domain), an NK activating domain that includes a fragment of IL-15, or an NK activating variant of IL-15 such as, for example, IL-15N72D or IL-15N72A, that includes an amino acid substitution compared to the wild-type IL-15 amino acid sequence.

The NK engaging domain and/or the targeting domain can include any moiety that is known to specifically bind to a target of interest. As described in more detail below, the NK engaging domain and/or the targeting domain can include an antibody, affibody, or other protein-based ligand (e.g., a Gp2-based protein ligand) known to bind to a selected target of interest. Once one selects a target of interest, a person of ordinary skill in the art can refer to databases and/or the scientific literature to identify suitable antibodies, affibodies, and/or protein-based ligands that specifically bind to the selected target of interest. As used herein, the term “selectively binds” refers to the ability to differentiate between two or more alternatives such as, for example, having differential affinity, to any degree, for a particular target. As used herein, the term “antibody” refers generally an immunoglobulin or a fragment thereof. Thus, as used herein, the term “antibody” encompasses antibody fragments capable of binding to a biological molecule (such as an antigen or receptor) or a portion thereof, including but not limited to Fab, Fab′ and F(ab′)₂, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody (e.g., a tribody) formed from antibody fragments. The antibody can be of any type (e.g., IgG, IgE, IgM, IgD, IgA and IgY), class (e.g., IgG1, IgG2, IgG3, IgG4, IgA1 and IgA2), or subclass.

Thus, in some embodiments, the NK cell engaging domain can include a humanized anti-CD16 antibody derived from an animal single domain antibody. While an scFv has a heavy variable chain component and a light variable chain component joined by a flanking sequence, a single domain antibody consists of a single monomeric variable chain—i.e., a variable heavy chin or a variable light chain—that is capable of specifically engaging a target. A single domain antibody may be derived from an antibody of any suitable animal such as, for example, a camelid (e.g., a llama or camel) or a cartilaginous fish. A single domain antibody can provide superior physical stability, an ability to bind deep grooves, and increased production yields compared to larger antibody fragments.

In one exemplary embodiment, a nanobody-based NK engager molecule can involve a humanized anti-CD16 single domain antibody derived from a llama nanobody (GeneBank sequence EF561291; Behar et al., 2008. Protein Eng Des Sel. 21(1): 1-10), termed EF91. Once humanized, the anti-CD16 single domain antibody (SEQ ID NO:3) can increase drug yield, increase stability, and/or increase NK-cell-mediated antibody-dependent cellular cytotoxicity (ADCC) efficacy of an IL-12 Killer Engager compound.

While described herein in the context of an exemplary embodiment in which the NK cell engaging domain includes a single-domain antibody (sdAb) that binds to CD16, an IL-12-based Killer Engager compound can include any other suitable NK engaging moiety. Exemplary alternative NK engaging moieties include, but are not limited to, any amino acid sequence (e.g., and antibody, an affibody, a protein-based ligand, or an NK-binding fragment of any of the foregoing) that can selectively bind to a receptor at least partially located at the surface of an NK cell. In certain embodiments, the NK cell engaging domain can selectively bind to a receptor that activates the NK cell and, therefore, also possess an activating function separate and apart from the NK activating domain. For example, activation of the CD16 receptor can elicit antibody-dependent cell-mediated cytotoxicity. Thus, the NK cell engaging domain of the exemplary IL-12 Killer Engager compounds described herein can possess NK activating activity. In other embodiments, the NK cell engaging domain may interrupt mechanisms that inhibit NK cells. In such embodiments, the NK cell engaging domain can include, for example, anti-PD-1/PD-L1, anti-NKG2A, anti-TIGIT, anti-killer-immunoglobulin receptor (KIR), and/or any other inhibition blocking domain.

The NK cell engaging domain can include an antibody or ligand that selectively binds to any NK cell receptor such as, for example, the cell cytotoxicity receptor 2B4, low affinity Fc receptor CD16, killer immunoglobulin like receptors (KIR), CD2, NKG2A, TIGIT, NKG2C, LIR-1, and/or DNAM-1.

One can design the NK cell engaging domain to possess a desired degree of NK selectivity and, therefore, a desired immune engaging character. For example, CD16 has been identified as Fc receptors FcγRIIIa (CD16a) and FcγRIIIb (CD16b). These receptors bind to the Fc portion of IgG antibodies that then activate the NK cell for antibody-dependent cell-mediated cytotoxicity. Anti-CD16 antibodies selectively bind to NK cells, but also can bind to neutrophils. Anti-CD16a antibodies selectively bind to NK cells, but do not bind to neutrophils. An IL-12-based Killer Engager compound that includes an NK cell engaging domain having an anti-CD16a antibody or CD16a-binding ligand can bind to NK cells but not bind to neutrophils. Thus, in circumstances where one may want to engage NK cells but not engage neutrophils, one can design the NK cell engaging domain of the IL-12-based Killer Engager compound to include an anti-CD16a antibody or a CD16a-binding ligand.

The targeting domain can include any moiety that selectively binds to an intended target such as, for example, a tumor cell, a target in the cancer stroma, a target on an inhibitory cell such as myeloid derived suppressor cells that are CD33″, or a target on a virally infected cell. Again, as discussed above, once one selects a target of interest, a person of ordinary skill in the art can refer to databases and/or the scientific literature to identify suitable antibodies and/or protein-based ligands that specifically bind to the selected target of interest.

Thus, a targeting domain can include, for example, an anti-tumor antibody and/or an anti-tumor ligand. Exemplary anti-tumor antibodies include, but are not limited to, rituximab (anti-CD20), afutuzumab (anti-CD20), trastuzumab (anti-HER2/neu), pertuzumab (anti-HER2/neu), labetuzumab (anti-CEA), adecatumumab (anti-EpCAM), citatuzumab bogatox (anti-EpCAM), edrecolomab (anti-EpCAM), arcitumomab (anti-CEA), bevacizumab (anti-VEGF-A), cetuximab (anti-EGFR), nimotuzumab (anti-EGFR), panitumumab (anti-EGFR), zalutumumab (anti-EGFR), gemtuzumab ozogamicin (anti-CD33), lintuzumab (anti-CD33), etaracizumab (anti-integrin αᵥβ₃), intetumumab (anti-CD51), ipilimumab (anti-CD152), oregovomab (anti-CA-125), votumumab (anti-tumor antigen CTAA16.88), pemtumumab (anti-MUC1), e23 (anti-HER2), lumretuzumab (anti-HER3), seribantumab (anti-HER3), KTN3379/CDX-3379 (anti-HER3), patritumab (anti-HER3), elgemtumab (anti-HER3), U3-1402 (anti-HER3), AV-203 (anti-HER3), GSK2849330 (anti-HER3), MM-111 (anti-HER3), MCLA-128 (anti-HER3), istiratumab (anti-HER3, antiIGFIR), duligotumab (anti-HER3), anti-CD19, anti-CD22, anti-CD133, anti-CD38, anti-B7H3, anti-PSMA, anti-mesothelin, anti-CLEC12A, anti-ROR1, anti-BCMA, anti-CSPG4, SS1, or anti-IGF1R.

Exemplary anti-tumor ligands include, but are not limited to, affibody or other protein-based ligands (e.g., Gp2-based ligands) that specifically bind to a tumor antigen.

Additional exemplary amino acid sequences suitable for use in the targeting domain are described in U.S. Pat. Nos. 11,098,100; 11,098,101; US Patent Application Publication No. 2020/0087369; and International Patent Publication Nos. WO 2021/055342, WO 2021/076545, and WO 2021/247794.

In other embodiments, the targeting domain can selectively bind to a target on a cell infected by a virus such as, for example, an adenovirus, HIV, CMV, and/or HPV.

In certain particular embodiments, the targeting domain can include an anti-CD33 antibody (SEQ ID NO:6) or an anti-CD33 protein ligand. In other particular embodiments, the targeting domain can include an anti-B7H3 antibody (SEQ ID NO:7) or an anti-B7H3 protein ligand. In other particular embodiments, the targeting domain can include an anti-epithelial cell adhesion molecule (EpCAM) antibody (SEQ ID NO:8) or an anti-EpCAM protein ligand.

In some embodiments, the molecule can further include a flanking sequence that can link two of the above-described domains. In some embodiments, the presence of the flanking sequence can further increase NK cell activation. Exemplary flanking sequences include the amino acid sequence of SEQ ID NOs: 12-23. Those of skill in the art can select an appropriate flanking sequence to offer a desired length and degree of flexibility to allow proper conformation of the Killer Engager compound.

Certain embodiments (e.g., the cam161233 Killer Engager, SEQ ID NO:1, and the cam1612B7H3 Killer Engagers, SEQ ID NO:2 and SEQ ID NO:10) can include more than one flanking sequence. As one example, the cam161233 Killer Engager of SEQ ID NO: 1 includes the flanking sequence of SEQ ID NO: 12 linking the NK engaging domain with the NK activating domain, and then again linking the IL-12A sequence with the IL-12B sequence within the NK activating domain. SEQ ID NO: 1 also includes the flanking sequence of SEQ ID NO:13 to link the NK activating domain with the targeting domain (e.g., anti-CD33 scFv). As another example, the cam1612B7H3 Killer Engager of SEQ ID NO: 10 includes the flanking sequence of SEQ ID NO:18 linking the NK engaging domain with the NK activating domain, the flanking sequence of SEQ ID NO: 12 linking the IL-12A sequence with the IL-12B sequence within the NK activating domain, and the flanking sequence of SEQ ID NO: 13 to link the NK activating domain with the anti-B7H3 targeting domain.

When more than one flanking sequence is include in an IL-12 Killer Engager compound, each flanking sequence can be selected independently of the particular flanking sequence used in any other flanking sequence within the compound.

This disclosure provides compositions and methods that involve IL-12-based NK Killer Engager compounds. Exemplary NK Killer Engager compounds include an IL-12-based NK activating domain that includes the amino acid sequence of SEQ ID NO:4, SEQ ID NO:5, or both.

Exemplary tri-specific Killer Engager compounds include the IL-12-based NK activating domain, an NK engaging domain, and a targeting domain. One such embodiment (SEQ ID NO:1) includes a camelid single domain antibody that specifically binds to CD16 (e.g., SEQ ID NO:3) as the NK engaging domain, human IL-12A (e.g., SEQ ID NO:5) and human IL-12B (e.g., SEQ ID NO:4) connected by a flanking sequence) as the NK activating domain, and an anti-CD33 antibody (e.g., SEQ ID NO:6) as the targeting domain. The entire amino acid sequence of this embodiment of Killer Engager compound is referred to herein as cam161233 (e.g., SEQ ID NO:1). Another tri-specific IL-12-based Killer Engager compound includes the camelid single domain antibody that specifically binds to CD16 (e.g., SEQ ID NO:3) as the NK engaging domain, human IL-12A (e.g., SEQ ID NO:5) and human IL-12B (e.g., SEQ ID NO:4) connected by a flanking sequence) as the NK activating domain, and an anti-B7H3 antibody (e.g., SEQ ID NO:7) as the targeting domain. The entire amino acid sequence of this embodiment of Killer Engager compound is referred to herein as cam1612B7H3 (e.g., SEQ ID NO:2). Yet another tri-specific IL-12-based Killer Engager compound is reflected in SEQ ID NO:10. In this embodiment, the NK engaging domain, NK activating domain, and targeting domain are the same as their counterpart domains in SEQ ID NO:2. However, the NK engaging domain, NK activating domain, and targeting domain in SEQ ID NO: 10 are linked by different flanking sequences than the flanking sequences that are used in SEQ ID NO:2.

An exemplary bi-specific Killer Engager compound includes the IL-12-based NK activating domain and an NK engaging domain. One such embodiment includes the camelid single domain antibody that specifically binds to CD16 (e.g., SEQ ID NO:3) as the NK engaging domain, human IL-12A (e.g., SEQ ID NO:5) and human IL-12B (e.g., SEQ ID NO:4) connected by a flanking sequence) as the NK activating domain.

Exemplary tetra-specific Killer Engager compounds can include the IL-12-based NK activating domain, an NK engaging domain, and two targeting domains. One such embodiment includes the camelid single domain antibody that specifically binds to CD16 (e.g., SEQ ID NO:3) as the NK engaging domain, human IL-12A (e.g., SEQ ID NO:5) and human IL-12B (e.g., SEQ ID NO:4) connected by a flanking sequence) as the NK activating domain, and two targeting domains. In some of these embodiments, the targeting domains can include an anti-CD33 antibody (e.g., SEQ ID NO:6) and an anti-CLEC12A antibody (e.g., SEQ ID NO:14) as the targeting domains. In other embodiments, the targeting domains can include an anti-B7H3 antibody (e.g., SEQ ID NO:7) and an antibody that specifically binds to, for example, PSMA, HER2, HER3, a HER2/HER3 complex, mesothelin, IGFIR, EpCAM (e.g., SEQ ID NO:8), or CD133.

Alternative exemplary tetra-specific Killer Engager compounds can include a targeting domain, an NK engaging domain, the IL-12-based NK activating domain, and a second NK activating domain. The second NK activating domain can include an anti-CD16 antibody, an anti-NKp46 antibody, an anti-NKp30 antibody, an anti-NKp40 antibody, an anti-NKG2D antibody, or an anti-NKG2C).

In all embodiments, whether, bi-specific, tri-specific, tetra-specific, or larger, the domains of the Killer Engager compound may be linked by flanking sequences, as described above.

Throughout this specification, reference to a particular amino acid sequence includes the identified amino acid sequence, but also includes functional variants of the identified amino acid sequence. As used herein, an amino acid sequence is a “functional variant” of a reference amino acid sequence if the “functional variant” amino acid sequence possesses a specified amount of sequence identity or sequence specificity compared to the reference amino acid sequence. An amino acid sequence is a “functional fragment” of a reference amino acid sequence if the “functional fragment” amino acid sequence contains less than the full-length amino acid sequence of the reference amino acid sequence. A “functional fragment” may further possess a specified amount of sequence identity or sequence specificity compared to the reference amino acid sequence.

Sequence similarity and/or sequence identity of two amino acid sequences can be determined by aligning the residues of the amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order.

A pair-wise comparison analysis of amino acid sequences can be carried out using the BESTFIT algorithm in the GCG package (version 10.2, Madison WI). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but allows for the presence of conservative substitutions. A conservative substitution for an amino acid residue within an amino acid sequence may be selected from other members of the class to which the amino acid residue belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Thus, conservative substitutions include, for example, Lys for Arg and vice versa to maintain a positive charge; Glu for Asp and vice versa to maintain a negative charge; Ser for Thr so that a free-OH is maintained; and Gln for Asn to maintain a free-NH₂.

Thus, an amino acid belonging to a grouping of amino acids having a particular size or characteristic (e.g., charge, hydrophobicity, or hydrophilicity) can be substituted for another amino acid without altering the activity of a protein, particularly in regions of the protein that are not directly associated with biological activity. Regions within an amino acid sequence that are not directly associated with biological activity may be deduced from alignment analyses, identifying regions where variability (e.g., additions, deletions, or non-conservative substitutions) are present when comparing related amino acid sequences. One can perform the alignment analysis using amino acid sequences provided herein and/or amino acid sequences readily available in databases.

An NK engaging domain, an NK activating domain, or a targeting domain can include an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to a reference amino acid sequence (e.g., a reference antibody fragment, a reference cytokine, or a reference cytokine fragment).

An NK engaging domain, an NK activating domain, or a targeting domain can include an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

An NK Killer Engager compound as described herein also can be designed to provide additional sequences, such as, for example, the addition of added C-terminal or N-terminal amino acids that can, for example, facilitate purification by trapping on columns or use of antibodies. Such tags include, for example, a histidine-rich tag (e.g., SEQ ID NO:11, with or without an additional spacer sequence) that allows one to purify polypeptides on nickel columns. Such gene modification techniques and suitable additional sequences are well known in the molecular biology arts.

In another aspect, this disclosure describes an isolated nucleic acid sequence that encodes any embodiment of an NK Killer Engager compound, or any component fragment of an NK Killer Engager compound, having the amino acid sequence of any one of SEQ ID NOs: 1-23. In some embodiments, the isolated nucleic acid encodes the NK Killer Engager compound of SEQ ID NO:1, SEQ ID NO:2, or SEQ ID NO:10. Given the amino acid sequence of any NK Killer Engager compound, or one or more component fragments of the NK Killer Engager compound, a person of ordinary skill in the art can determine the full scope of polynucleotides that encode that amino acid sequence using conventional, routine methods.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A nucleic acid may be single-stranded, double-stranded, linear, or covalently circularly closed molecule. A nucleic acid can be isolated. The term “isolated nucleic acid” means that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be introduced—i.e., transfected-into cells. When RNA is used to transfect cells, the RNA may be modified by stabilizing modifications, capping, or polyadenylation.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, which relies on agarose gel electrophoresis, polyacrylamide gel electrophoresis, or high-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; and 9,012,208. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Separated molecules may be sequenced by sequential or single extension reactions using polymerases or ligases as well as by single or sequential differential hybridizations with libraries of probes.

In another aspect, this disclosure describes a host cell including any of the isolated nucleic acid sequences and/or proteins described herein. Thus, this disclosure encompasses translation of a nucleic acid (e.g., an mRNA) by a host cell to produce an NK Killer Engager compound.

The nucleic acid constructs of the present invention may be introduced into a host cell to be altered, thus allowing expression of the NK Killer Engager compound within the cell, thereby generating a genetically engineered cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE (Thermo Fisher Scientific, Inc., Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc USA, San Diego, CA).

The nucleic acid constructs described herein may be introduced into a host cell to be altered, thus allowing expression within the cell of the protein encoded by the nucleic acid. A variety of host cells are known in the art and suitable for protein expression. Examples of typical cell used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

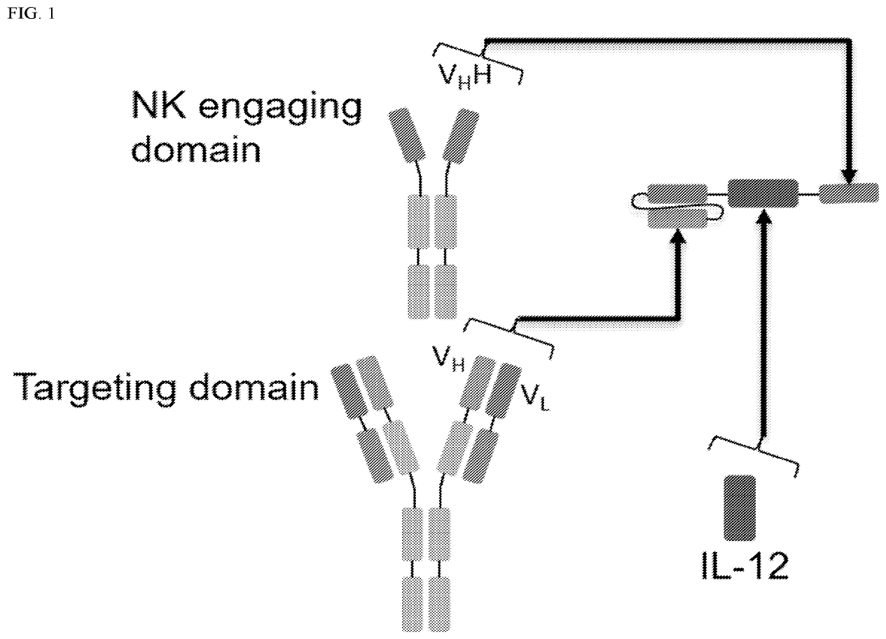

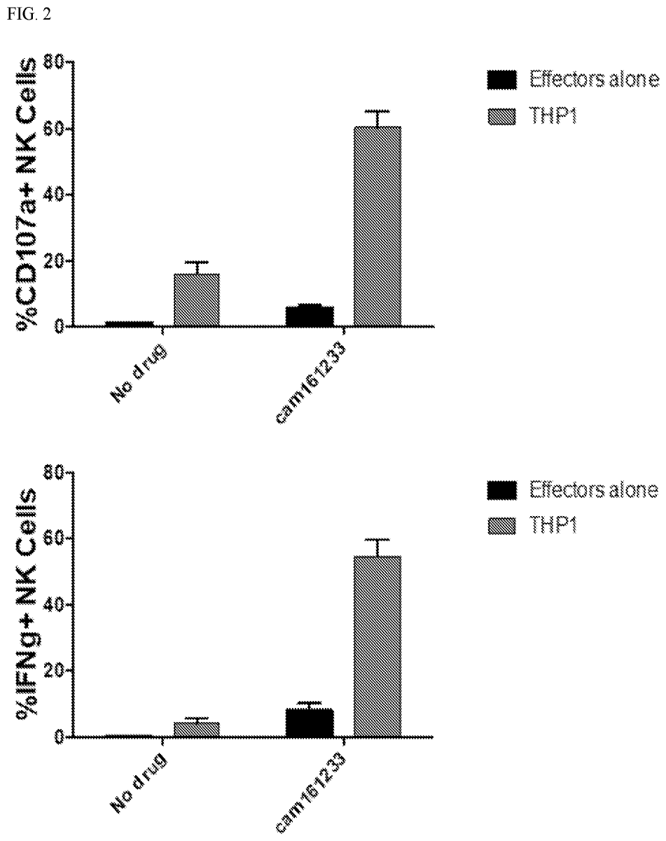

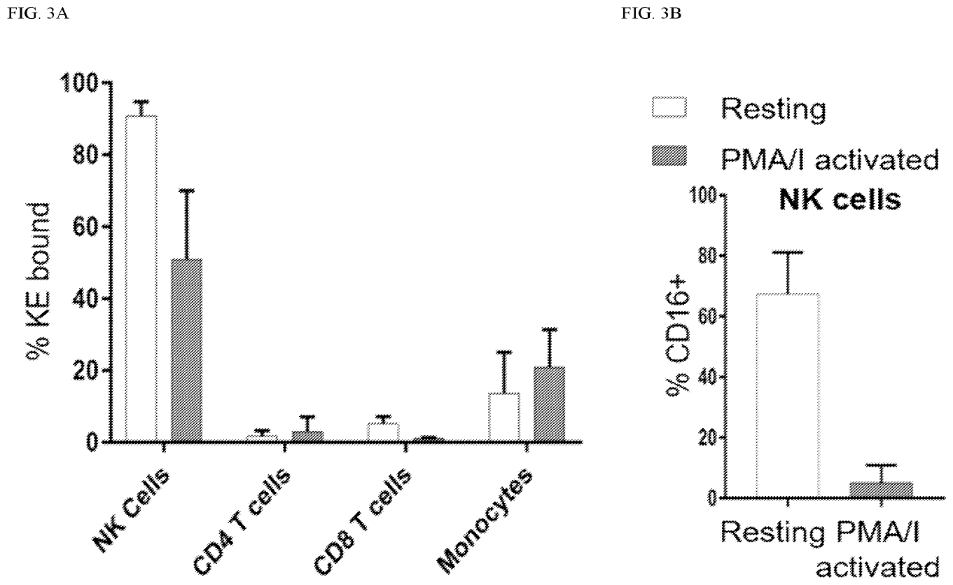

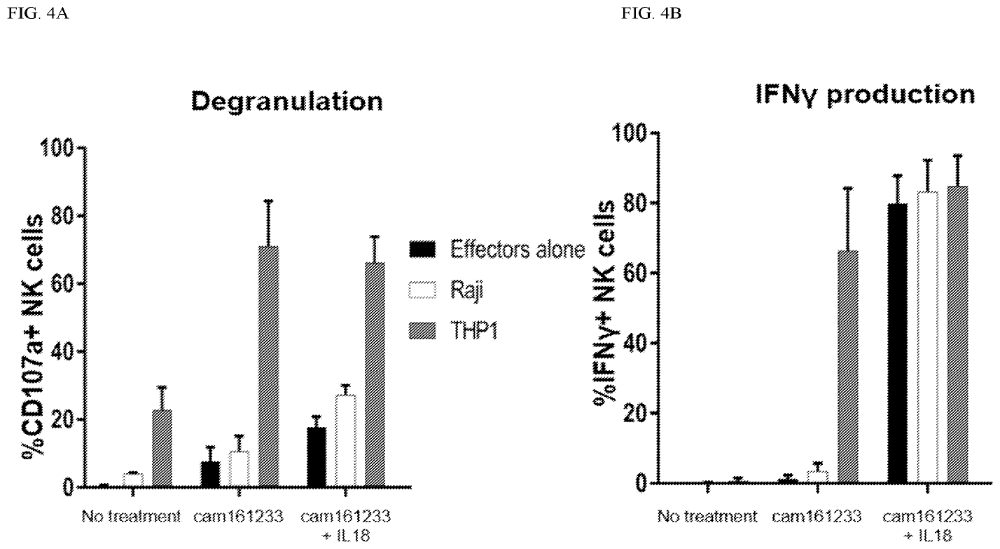

IL-12 is a potent interferon-gamma (IFNγ) inducer for NK cells and reduces tumor burden in preclinical models. However, when tested clinically, it was found to be overly toxic. Targeted delivery of IL-12 within an NK cell Killer Engager compound decreases toxicity of IL-12. An exemplary IL-12-based Killer Engager compound (cam161233; SEQ ID NO:1) includes an anti-CD16 NK engaging domain, an IL-12-based NK activating domain, and an anti-CD33 targeting domain. The model NK Killer Engager compound is shown schematically in FIG. 1. In vitro, the model IL-12-based NK Killer Engager cam161233 (SEQ ID NO:1) induced NK cell degranulation against CD33″ targets (FIG. 4A) and IFNγ production (FIG. 4B) at substantially higher levels and had a more restricted cytokine repertoire than NK cells incubated with a comparable IL-15-based Killer Engager (cam161533; SEQ ID NO:24). The IL-15-based Killer Engager induced higher levels of cytokine storm, inducing cytokines such as IL-6. FIG. 4 shows that while the cam161233 is functional and specific (FIG. 4A,B, cam161233 alone), the data from treatment with cam161233+IL-18 indicates that the activity seen is not only mediated by crosslinking of CD16/CD33, but also due to functional IL-12 signaling by the NK activating domain.

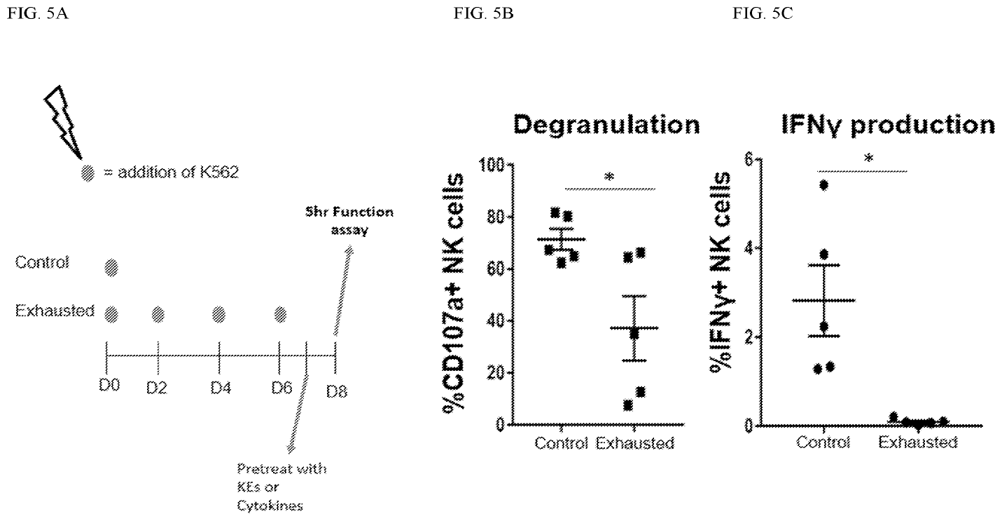

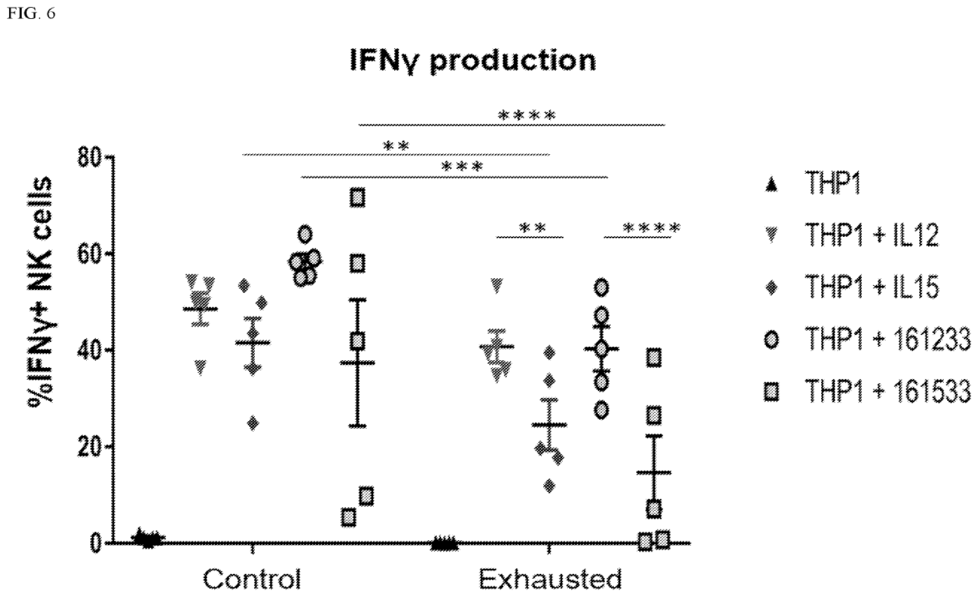

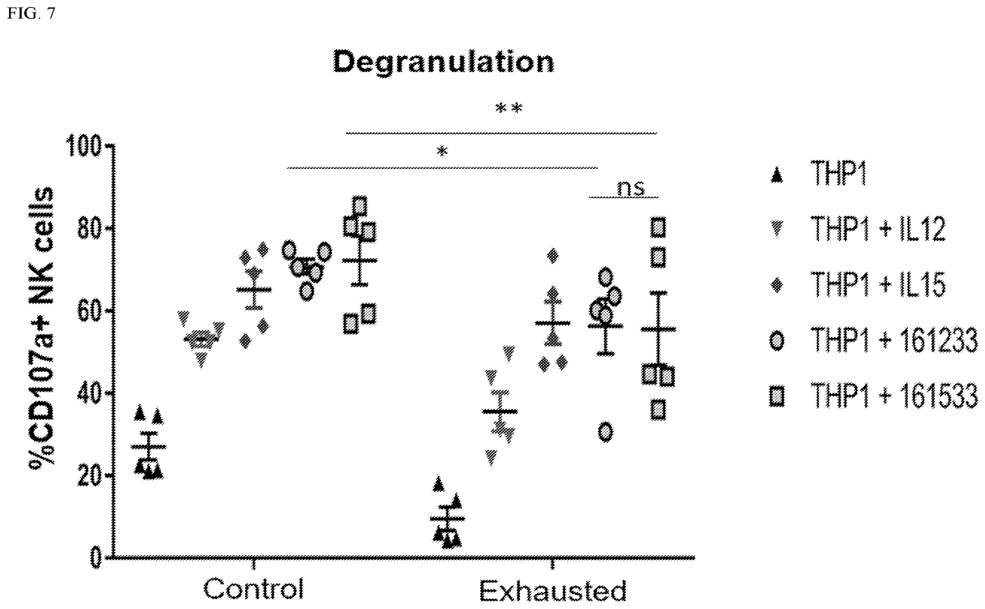

IL-12 can rescue T cells and NK cells from exhaustion. A model of NK cell exhaustion using repeated tumor stimulation was used to examine the ability of the IL-12-based Killer Engager to rescue NK cells from exhaustion. FIGS. 5-7 show that the model IL-12-based Killer Engager cam161233 (SEQ ID NO:1) rescued degranulation and IFNγ production in this model.

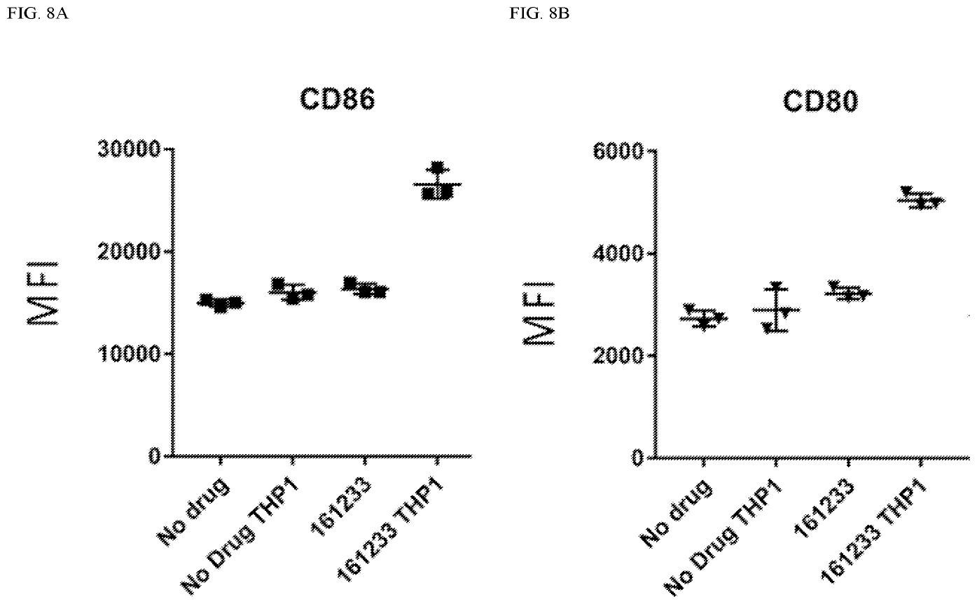

Another feature of the IL-12-based Killer Engager is its ability to induce dendritic cell maturation, further enhancing the immune response. In FIG. 8, NK cells were incubated with CD33⁺ THP1 targets and the noted treatments and then the supernatant from those cultures was added to immature monocyte-derived dendritic cells to evaluate whether the IL-12 Killer Engager compound can alter the milieu of NK-secreted cytokines to enhance NK cell maturation. The model IL-12-based Killer Engager cam161233 (SEQ ID NO: 1) can induce upregulation of CD80 and CD86, two markers of dendritic cell maturation. This enhancement of dendritic cell maturation enhances the adaptive immune response against the target (e.g., a tumor).

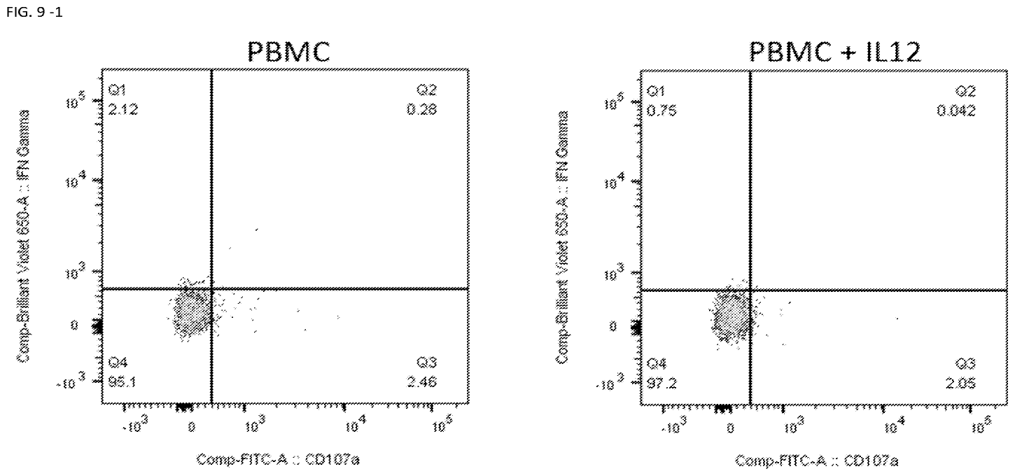

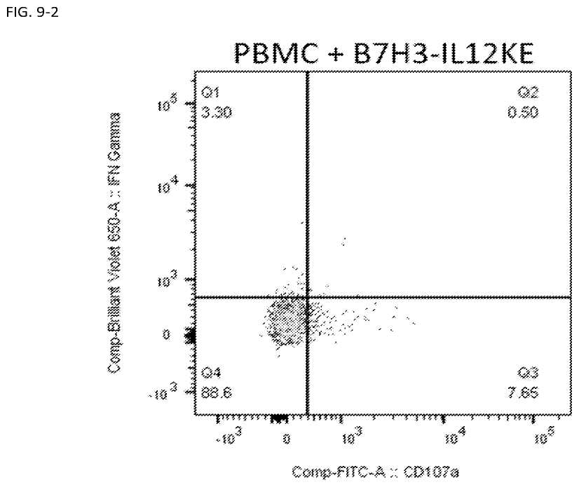

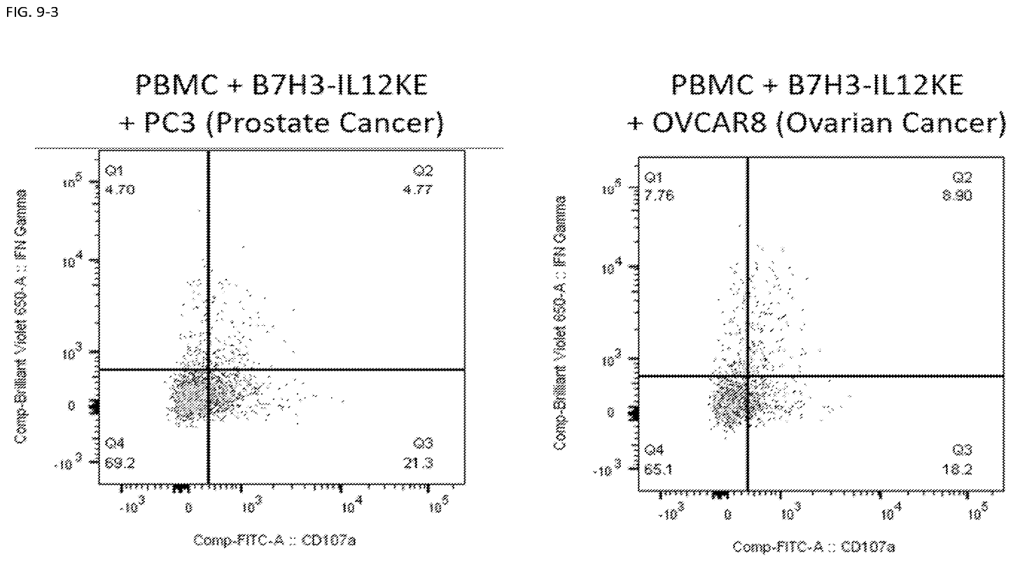

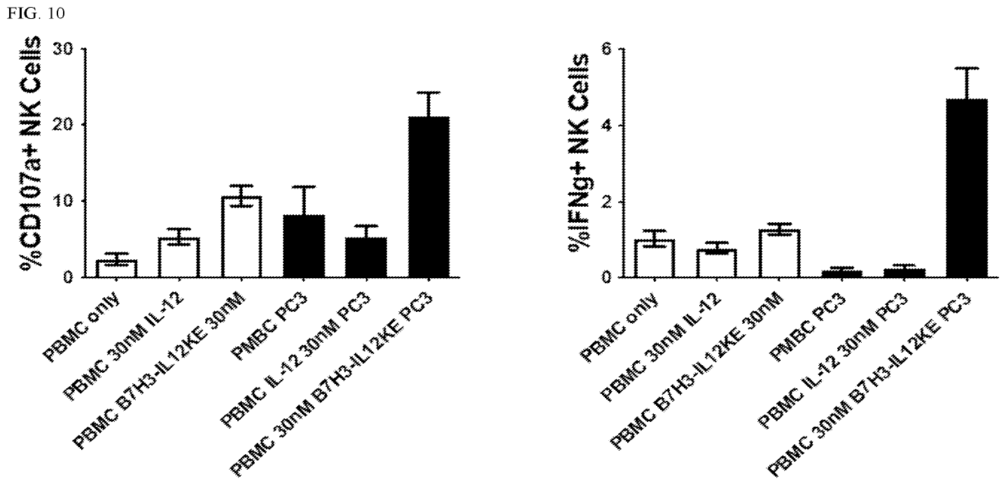

FIG. 9 and FIG. 10 provide data generated using a different model IL-12-based Killer Engager compound (cam1612B7H3 (B7H3-IL12KE); SEQ ID NO:10). FIG. 9 shows that the model IL-12 Killer Engager cam1612B7H3 (SEQ ID NO:10) induces NK cell activation against solid tumors. To measure activation, surface CD107a (a measure of NK cell degranulation) and intracellular interferon gamma (IFNγ; a measure of NK cell inflammatory cytokine production) were evaluated by flow cytometry on gated CD56 CD3″ NK cells. FIG. 10 shows pooled data showing that the model IL-12 Killer Engager (cam1612B7H3 (B7H3-IL12KE); SEQ ID NO:10) induces NK cell activation against a model tumor cell line, prostate cancer cell line PC3. To measure activation, surface CD107a (a measure of NK cell degranulation; left) and intracellular interferon gamma (IFNγ; a measure of NK cell inflammatory cytokine production; right) was evaluated by flow cytometry on gated CD56 CD3″NK cells.

In another aspect, this disclosure describes methods of killing a target cell in a subject. Generally, the method includes administering to the subject an IL-12-based Killer Engager compound in an amount effective to induce NK-mediated killing of the target cells. “Treat” or variations thereof refer to reducing, limiting progression, ameliorating, or resolving, to any extent, the symptoms or signs related to a condition. As used herein, “ameliorate” refers to any reduction in the extent, severity, frequency, and/or likelihood of a symptom or clinical sign characteristic of a particular condition; “symptom” refers to any subjective evidence of disease or of a patient's condition; and “sign” or “clinical sign” refers to an objective physical finding relating to a particular condition capable of being found by one other than the patient.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Thus, in certain embodiments, the method can involve prophylactic treatment of a subject at risk of developing a condition. “At risk” refers to a subject that may or may not actually possess the described risk. Thus, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition. Exemplary indicia of a condition can include, for example, genetic predisposition, ancestry, age, sex, geographical location, lifestyle, or medical history. Treatment may also be continued after symptoms have resolved, for example to prevent or delay their recurrence.

In some cases, the treatment can involve administering the IL-12-based Killer Engager compound to a subject so that the IL-12-based Killer Engager compound can stimulate endogenous NK cells in vivo. Using an IL-12-based Killer Engager compound as a part of an in vivo method can make NK cells antigen specific with simultaneous co-stimulation, enhancement of survival, and expansion, which may be antigen specific. In other cases, the IL-12-based Killer Engager can be used in vitro as an adjuvant to NK cell adoptive transfer therapy.

Accordingly, an IL-12-based Killer Engager compound may be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the IL-12-based Killer Engager compound is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

The IL-12-based Killer Engager compound can be any embodiment of the IL-12-based Killer Engager compound described above having a targeting domain that selectively binds to an appropriate target cell population. In some cases, the target cell can include a tumor cell so that the method involves treating cancer associated with the tumor cells. Thus, in some embodiments, the method can include ameliorating at least one symptom or clinical sign of the tumor.

In embodiments in which the target cell includes a tumor cell, the method can further include surgically resecting the tumor and/or reducing the size of the tumor through chemical (e.g., chemotherapeutic) and/or radiation therapy. Exemplary tumors that may be treated include tumors associated with prostate cancer, lung cancer, mesothelioma, colon cancer, rectum cancer, urinary bladder cancer, melanoma, kidney cancer, renal cancer, oral cavity cancer, pharynx cancer, pancreas cancer, uterine cancer, thyroid cancer, skin cancer, head and neck cancer, cervical cancer, ovarian cancer and/or hematopoietic cancer.

In various embodiments, the IL-12-based Killer Engager targeting domain can include a polypeptide that selectively binds to, for example, EGFR, HER2/neu, EpCAM, CSPG4, HSPG2, IGF-1, CD38, CD19, CD20, CD22, CD30, CD52, CD33, ROR-1, UPAR, VEGFR, LIV-1, SGN-CD70A, CD70, IL-3, IL-4R, CD133, MUC-1, B7H3, PSMA, CLEC12A, BCMA, mesothelin, the epithelial-mesenchymal transition (EMT), TRAIL, CD38, CD45, CD74, CD23, or cancer viral markers such as HIV.

As used herein, a “subject” can be any animal such as, for example, a mammal (e.g., dog, cat, horse, cow, sheep, goat, monkey, other mammalian companion or livestock animal, etc.). In certain embodiments, the subject can be a human.

An IL-12-based Killer Engager compound described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with an IL-12-based Killer Engager compound without causing any undesirable biological effects or interacting in a deleterious manner with any of the other components of the pharmaceutical composition in which it is contained.

An IL-12-based Killer Engager compound may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release. In certain embodiments, the composition is administered intraperitoneally, intravenously, or subcutaneously.

Thus, an IL-12-based Killer Engager compound may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like. In certain embodiments, the composition may be formulated into a solution or a suspension.

A formulation may be conveniently presented in unit dosage form and may be prepared by methods well known in the art of pharmacy. Methods of preparing a composition with a pharmaceutically acceptable carrier include the step of bringing an IL-12-based Killer Engager compound into association with a carrier that constitutes one or more accessory ingredients. In general, a formulation may be prepared by uniformly and/or intimately bringing the active molecule into association with a liquid carrier, a finely divided solid carrier, or both, and then, if necessary, shaping the product into the desired formulations.

The amount of IL-12-based Killer Engager compound administered can vary depending on various factors including, but not limited to, the specific IL-12-based Killer Engager compound being used, the weight, physical condition, and/or age of the subject, and/or the route of administration. Thus, the absolute weight of IL-12-based Killer Engager compound included in a given unit dosage form can vary widely, and depends upon factors such as the species, age, weight, and physical condition of the subject, and/or the method of administration. Accordingly, it is not practical to set forth generally the amount that constitutes an amount of IL-12-based Killer Engager compound effective for all possible applications. Those of ordinary skill in the art, however, can readily determine the appropriate amount with due consideration of such factors.

In some embodiments, the method can include administering sufficient IL-12-based Killer Engager compound to provide a dose of, for example, from about 100 ng/kg to about 10 mg/kg to the subject, although in some embodiments the methods may be performed by administering IL-12-based Killer Engager compound in a dose outside this range.

In some embodiments, the method can includes administering sufficient IL-12-based Killer Engager compound to provide a minimum dose of at least 100 ng/kg/day such as, for example, at least 1 μg/kg/day, at least 5 μg/kg/day, at least 10 μg/kg/day, at least 25 μg/kg/day, at least 50 μg/kg/day, at least 100 μg/kg/day, at least 200 μg/kg/day, at least 300 μg/kg/day, at least 400 μg/kg/day, at least 500 μg/kg/day, at least 600 μg/kg/day, at least 700 μg/kg/day, at least 800 μg/kg/day, at least 900 μg/kg/day, or at least 1 mg/kg/day.

In some embodiments, the method includes administering sufficient IL-12-based Killer Engager compound to provide a maximum dose of no more than 10 mg/kg/day such as, for example, no more than 5 mg/kg/day, no more than 4 mg/kg/day, no more than 3 mg/kg/day, no more than 2 mg/kg/day, no more than 1 mg/kg/day, no more than 900 μg/kg/day, no more than 800 μg/kg/day, no more than 700 μg/kg/day, no more than 600 μg/kg/day, no more than 500 μg/kg/day, no more than 400 μg/kg/day, no more than 300 μg/kg/day, no more than 200 μg/kg/day, no more than 100 μg/kg/day, no more than 90 μg/kg/day, no more than 80 μg/kg/day, no more than 70 μg/kg/day, no more than 60 μg/kg/day, no more than 50 μg/kg/day, no more than 40 μg/kg/day, no more than 30 μg/kg/day, no more than 20 μg/kg/day, or no more than 10 μg/kg/day. The IL-12-based Killer Engager compound provides a dose of “no greater than” a specified amount when the IL-12-based Killer Engager compound is not absent but is present in an amount up to and including the specified amount.

In some embodiments, the method includes administering sufficient IL-12-based Killer Engager compound to provide a dose characterized by a range having endpoints defined by any a minimum dose identified above and any maximum dose that is greater than the selected minimum dose. For example, in some embodiments, the method can include administering sufficient IL-12-based Killer Engager compound to provide a dose of from about 10 μg/kg/day to about 10 mg/kg/day to the subject, a dose of from about 100 μg/kg/day to about 1 mg/kg/day, a dose of from 5 μg/kg/day to 100 μg/kg/day, etc.

In certain embodiments, the method includes administering sufficient IL-12-based Killer Engager compound to provide a dose that is equal to any minimum dose or any maximum dose listed above. Thus, for example, in certain embodiments, the method can include administering sufficient IL-12-based Killer Engager compound to provide a dose of 1 μg/kg/day, 5 μg/kg/day, 10 μg/kg/day, 25 μg/kg/day, 50 μg/kg/day, 100 μg/kg/day, 200 μg/kg/day, 500 μg/kg/day, 1 mg/kg/day, 5 mg/kg/day, etc.

In some embodiments, an IL-12-based Killer Engager compound may be administered, for example, from a single dose to multiple doses per week, although in some embodiments the method can be performed by administering an IL-12-based Killer Engager compound at a frequency outside this range. In certain embodiments, an IL-12-based Killer Engager compound may be administered from about once per month to about five times per week. In some embodiments, the doses indicated above, which are described in terms of the amount of IL-12-based Killer Engager compound administered over a 24-hour period, are administered in a seven-day cycle of four days of treatment and three days of rest.

In some embodiments, an IL-12-based Killer Engager compound may be administered, for example, from a single dose to multiple cycles of treatment, although in some embodiments the method can be performed by administering an IL-12-based Killer Engager compound for a duration outside this range. In some embodiments, the IL-12-based Killer Engager compound may be administered for three weeks. In such embodiments, each week may be a treatment cycle such as the exemplary treatment cycle described in the preceding paragraph. In other embodiments, the IL-12-based Killer Engager compound may be administered for a greater number of treatment cycles, without a gap between one set of treatment cycles and a subsequent set of treatment cycles. The gap between one set of treatment cycles and a subsequent set of treatment cycles may be a gap of one or more weeks, one or more months, or one or more years.

In some embodiments, the method further includes administering one or more additional therapeutic agents. The one or more additional therapeutic agents may be administered before, after, and/or coincident to the administration of an IL-12-based Killer Engager compound. An IL-12-based Killer Engager compound and the additional therapeutic agents may be co-administered. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another. When administered sequentially, any component may be administered prior to any other component. In other embodiments, the IL-12-based Killer Engager compound and the additional therapeutic agent may be administered as part of a mixture or cocktail. In some aspects, the administration of IL-12-based Killer Engager compound may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

Exemplary additional therapeutic agents can include chemotherapy agents. Suitable additional therapeutic agents include, but are not limited to, altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, vinorelbine, an anti-HER2 antibody therapy, an anti-HER3 antibody therapy (see, e.g., Liu et al., 2019, Biol Proced Online 21:5), or an anti-HER2/HER3 heterodimer complex antibody therapy (see, e.g., Liu et al., 2019, Biol Proced Online 21:5).

In some embodiments, the method can include administering sufficient IL-12-based Killer Engager compound as described herein and administering the at least one additional therapeutic agent in respective amounts effective to demonstrate therapeutic synergy. In some aspects of the methods of the present invention, a measurement of response to treatment observed after administering both an IL-12-based Killer Engager compound as described herein and the additional therapeutic agent is improved over the same measurement of response to treatment observed after administering either the IL-12-based Killer Engager compound or the additional therapeutic agent alone. In some embodiments, an additional therapeutic agent can include an additional agent that targets EpCAM including, for example, an EpCAM specific monoclonal antibody, such as, for example, catumaxomab, a monoclonal hybrid antibody targeting EpCAM and CD3.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Function Assay Measuring CD107a Degranulation and IFNγ Cytokine Production

Flow cytometric assessment of NK-cell function was carried out as described previously (Felices et al., Blood Adv 3:897-907; 2019). Briefly, following addition of noted treatments [diluted in RPMI1640 (GIBCO, Thermo Fisher Scientific, Inc., Waltham, MA) supplemented with 10% FBS (GIBCO, Thermo Fisher Scientific, Inc., Waltham, MA)] and HL-60 (cam161233 or CD33-IL-12KE) or OVCAR8/PC3 (cam1612B7H3 or B7H3-IL12KE) tumor targets [at a 2:1 effector:target (E:T) ratio], healthy donor or posttransplant patient PBMCs were stained with FITC-conjugated anti-CD107a (BioLegend, San Diego, CA) at the beginning of a four-hour incubation. One hour after the addition of anti-CD107a, cells were given Golgi Stop (1:1,500) and Golgi Plug (1:1,000; both from BD Biosciences, San Jose, CA), and incubated for three hours. Cells were then stained with the Live/Dead Fixable Aqua Staining Kit (Thermo Fisher Scientific, Inc., Waltham, MA), anti-CD56, and anti-CD3, fixed in 2% paraformaldehyde (Thermo Fisher Scientific, Inc., Waltham, MA), and permeabilized with permeabilization buffer (eBioscience, San Diego, CA). Permeabilized cells were stained with BV65 (BioLegend, San Diego, CA).

### NK Cell Binding

Briefly, cells are treated with media (RPMI-1640 with 10% fetal bovine serum) alone or PMA/I (20 ng/ml and 1 μg/ml (respectively) for one hour, then washed twice with media, then incubated with cam161233 (30 nM) for 30 minutes, followed by two washes, followed by incubation with a phycoerythrin (PE)-labeled anti-HIS antibody (the IL-12 Killer Engager research molecule has a 10×-HIS tag), followed by surface antibody staining (CD56, CD3, CD8, and CD14). Cells are then fixed and subjected to flow cytometry (LSR II, BD Biosciences, San Jose, CA) to analyze binding.

### NK Exhaustion Assay

Briefly, K562 (CML line that potently activates natural cytotoxicity on NK cells) cells were added at multiple times over the course of eight days. On the eighth day, effectors (NK cells) were incubated with K562 targets at a 2:1 ratio. After five hours, cells were stained for degranulation (CD107a) and inflammatory (IFNγ) markers and subjected to flow cytometry (LSR II, BD Biosciences, San Jose, CA). Data was analyzed using FLOWJO software (BD Biosciences, San Jose, CA).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A compound comprising:
an NK cell engaging domain that selectively binds to an NK cell;
an NK activating domain operably linked to the NK cell engaging domain comprising IL-12 or a functional fragment thereof;
a first flanking sequence linking the NK cell engaging domain with the NK activating domain;
a targeting domain that selectively binds to a target antigen; and
a second flanking sequence linking the NK activating domain and the targeting domain.

2. (canceled)

3. The compound of claim 1, wherein the NK cell engaging domain comprises an antibody or a binding fragment thereof.

4. (canceled)

5. (canceled)

6. The compound of claim 1, wherein the NK engaging domain comprises an affibody or a Gp2-based protein ligand.

7. The compound of claim 1, wherein the IL-12 comprises:
an IL-12A sequence;
an IL-12B sequence; or
an IL-12A sequence and an IL-12B sequence.

8-11. (canceled)

12. The compound of claim 1, wherein the targeting domain comprises an antibody or a binding fragment thereof.

13. The compound of claim 12, wherein the antibody fragment comprises an scFv, a F(ab′)2, a Fab, or a single-domain antibody fragment.

14. (canceled)

15. (canceled)

16. The compound of claim 1, wherein the targeting domain comprises an affibody or a Gp2-based protein ligand.

17. The compound of claim 1, wherein the first flanking sequence comprises the amino acids of any one of SEQ ID NO: 12-23.

18. The compound of claim 1, wherein the second flanking sequence comprises the amino acids of any one of SEQ ID NO: 12-23.

19. The compound of claim 1, further comprising a second targeting domain.

20. (canceled)

21. (canceled)

22. A composition comprising:
the compound of claim 1; and
a pharmaceutically acceptable carrier.

23. The composition of claim 22, further comprising an additional therapeutic agent.

24. (canceled)

25. A method comprising:
administering to a subject the compound of claim 1 in an amount effective to induce NK-mediated killing of a cancer cell.

26. A method for stimulating degranulation of NK cells in vivo, the method comprising:
administering to a subject an amount of the compound of claim 1 effective to stimulate degranulation of NK cells in the subject.

27. A method for stimulating IFNγ secretion by NK cells in vivo, the method comprising:
administering to a subject an amount of the compound of claim 1 effective to stimulate IFNγ secretion by of NK cells in the subject.

28. A method of rescuing exhaustion of NK cells in vivo, the method comprising:
administering to a subject an amount of the compound of claim 1 effective to rescue exhaustion by of NK cells in the subject.

29. A method of treating cancer in a subject, the method comprising:
administering to the subject an amount of the compound of claim 1 effective for treating the cancer.

30. The method of claim 29, further comprising administering the compound prior to, simultaneously with, or following chemotherapy, surgical resection of a tumor, or radiation therapy.

31. (canceled)

32. A method comprising:
administering to a subject the composition of claim 22 in an amount effective to induce NK-mediated killing of a cancer cell.

33-35. (canceled)

36. A method of treating cancer in a subject, the method comprising:
administering to the subject an amount of the composition of claim 22 effective for treating the cancer.

37. (canceled)

38. (canceled)

39. A method comprising:
administering to a subject the composition of claim 23 in an amount effective to induce NK-mediated killing of a cancer cell.

40. A method of treating cancer in a subject, the method comprising:
administering to the subject an amount of the composition of claim 23 effective for treating the cancer.


SEQUENCE LISTING (24 sequences)

SEQ ID NO: 1 - protein, 915 aa, Artificial
  Polypeptide
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSTSGSGKPGSGEGSTKGQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 2 - protein, 795 aa, Artificial
  Polypeptide
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSTSGSGKPGSGEGSTKGQVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 3 - protein, 122 aa, Artificial
  Polypeptide
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 4 - protein, 306 aa, Artificial
  Polypeptide
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 197 aa, Artificial
  Polypeptide
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 242 aa, Artificial
  Polypeptide
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 7 - protein, 122 aa, Artificial
  Polypeptide
QVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 8 - protein, 260 aa, Artificial
  Polypeptide
EASGGPEDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRATPSHNSHQVPSAGGPTANSGTSGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 9 - protein, 123 aa, Artificial
  Polypeptide
QVQLLESGGGLVQPGGSLRLSCAASGDMFSYDDMGWVRQAPGKGLEWVSGIQNTDGSTYYADSVKGRFTISRDNSKNTLYLQMNSLRAEDTAVYYCATLYDRMVGKEEQLASWGQGTLVTVSS

SEQ ID NO: 10 - protein, 802 aa, Artificial
  Polypeptide
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSTSGSGKPGSGEGSTKGQVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 11 - protein, 10 aa, Artificial
  Polypeptide
HHHHHHHHHH

SEQ ID NO: 12 - protein, 15 aa, Artificial
  Polypeptide
GGGGSGGGGSGGGGS

SEQ ID NO: 13 - protein, 18 aa, Artificial
  Polypeptide
GSTSGSGKPGSGEGSTKG

SEQ ID NO: 14 - protein, 20 aa, Artificial
  Polypeptide
PSGQAGAAASESLFVSNHAY

SEQ ID NO: 15 - protein, 7 aa, Artificial
  Polypeptide
EASGGPE

SEQ ID NO: 16 - protein, 38 aa, Artificial
  Polypeptide
AEAAKEAAKEAAKEAAKALEAEAAKEAAKEAAKEAAKA

SEQ ID NO: 17 - protein, 10 aa, Artificial
  Polypeptide
AEAAKEAAKA

SEQ ID NO: 18 - protein, 22 aa, Artificial
  Polypeptide
SGGGGSGGGGSGGGGSGGGGSG

SEQ ID NO: 19 - protein, 10 aa, Artificial
  Polypeptide
GGGGSGGGGS

SEQ ID NO: 20 - protein, 18 aa, Artificial
  Polypeptide
GSTSGSGKPGSGEGSTKG

SEQ ID NO: 21 - protein, 15 aa, Artificial
  Polypeptide
EPKSSDKTHTSPPSP

SEQ ID NO: 22 - protein, 25 aa, Artificial
  Polypeptide
RATPSHNSHQVPSAGGPTANSGTSG

SEQ ID NO: 23 - protein, 19 aa, Artificial
  Polypeptide
SSGGGGSGGGGGGSSRSSL

SEQ ID NO: 24 - protein, 507 aa, Artificial
  Polypeptide
MEQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSPSGQAGAAASESLFVSNHAYNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEASGGPEQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK